(19) United States
(12) Patent Application Publication
(10) Pub. No.: US 2007/0219171 A1
(43) Pub. Date: Sep. 20, 2007

(54) Transdermal Pharmaceutical Spray Formulations Comprising a Vp/Va Copolymer and a Non-Aquous Vehicle

(75) Inventors: Amar Lulla, Maharashtra (IN); Geena Malhotra, Maharashtra (IN)

Correspondence Address: CONLEY ROSE, P.C., 5700 GRANITE PARKWAY, SUITE 330, PLANO, TX, 75024, US

(73) Assignee: Waterlead Limited, Douglas, Isle of Man (ZA)

(21) Appl. No.: 10/576,908

(22) PCT Filed: Oct. 21, 2004
(86) PCT No.: PCT/GB04/04487

§ 371 Date: Dec. 18, 2006

(30) Foreign Application Priority Data
Oct. 23, 2003 (IN) 1122/MUM/2003

(51) Int. Cl. A61K 31/568; A61K 31/565; A61K 47/32

(52) U.S. Cl. 514/177; 514/182; 514/772.5

(57) ABSTRACT

Transdermal spray formulations containing a pharmaceutically active agent and methods for administering the same are provided. The formulations comprise the pharmaceutically active agent, a VP/VA copolymer and a non-aqueous vehicle. The formulations further comprise an anti-nucleating agent to prevent recrystallization of the pharmaceutically active agent and a penetration enhancer to increase the rate of drug delivery through the skin. Upon application to the skin, the present formulations dry to provide a film at the site of treatment.

### CROSS-REFERENCE TO RELATED APPLICATIONS

This application is a filing under 35 U.S.C. 371 of International Application No. PCT/GB2004/004487 filed Oct. 21, 2004, entitled “Transdermal Pharmaceutical Spray Formulations Comprising A VP/VA Copolymer And A Non-Aqueous Vehicle,” claiming priority of Indian Patent Application No. IN 1122/MUM/2003 filed Oct. 23, 2003, which applications are incorporated by reference herein in their entirety.

### FIELD OF THE INVENTION

The invention relates generally to transdermal drug delivery formulations. More specifically, the invention relates to spray formulations for delivering a pharmaceutically active agent to the skin. Any drug suitable for transdermal, transcutaneous or topical administration, including local and systemic active agents, can be used in the present formulations.

### BACKGROUND OF THE INVENTION

When technically feasible, topical or transdermal delivery of drugs for both local and systemic indications offers many advantages over oral administration. Benefits of transdermal delivery include increased patient compliance, localized drug targeting, control over rate of absorption and avoidance of reduced bioavailability due to first pass metabolism effects in the liver. Classic topical delivery vehicles include ointments, creams, lotions, pastes and gels.

More recently, controlled-release topical patches have become available. Topical patches are capable of delivering active substances to the skin in a controlled, sustained-release manner and have been shown to be effective in the long-term delivery of sustained therapeutic levels of active substances.

Prior art does exist in the fields of external preparations for topical administration and transdermal patches. EP 0812588 describes such a preparation which aims at inhibiting rejection reactions at organ transplantation or treating autoimmune diseases or allergic diseases.

A transdermal patch for administering a volatile liquid drug such as nicotine transdermally to a patient is described in Patent Application No. WO 0033812.

WO 03035510 discloses a dispenser for conveniently dispensing multiple transdermal transmucosal drug-containing patches from a single container.

Emu-oil based formulations in the form of a spray or transdermal formula for use as an analgesic, anaesthetic and antipruritic are described in U.S. Pat. No. 6,528,040.

Transdermal patch and topical compositions containing propylnorapomorphine are disclosed in EP Patent Application No. 1098637 and related applications.

Patent Application No. JP 2002 84701 describes a patch for topical treatment of acne. A topical patch preparation containing a delayed-type hypersensitivity inducer and methods for using the same are disclosed in Patent Application No. WO 02072081. A topical anesthetic patch is also described in U.S. Pat. No. 6,274,167.

Patent Application No. WO 0137890 describes a propellant-free spray-on skin patch composition for improving wound healing and for drug administration. EP 560014, EP 6400352 and EP 409550 are among the main prior art documents cited in the search report of the Patent Application No. WO 0137890.

The above prior art indicates the recent increased attention in transdermal patches, however, topical patches can be relatively expensive to produce, and often exhibit reduced adhesion to the skin over time. Irritation has been known to result from patch removal or from adhesive residues left on the skin. Moreover, after use, patches require that appropriate measures be taken to assure safe disposal in order to prevent danger to children or animals.

A number of topical formulations for transdermal delivery of pharmaceuticals have been proposed. However, each of these prior formulations are substantially aqueous solutions and are limited in that they are only suitable for the delivery of water-soluble drugs. Moreover, although they form non-flowing gels that adhere to skin at body temperature, said gels remain wet to the touch on the skin and can be easily wiped away unless covered with a dressing, thereby requiring the subject to avoid contact with the treated area.

### SUMMARY OF THE INVENTION

The present invention overcomes or alleviates the problems of the prior art.

In a first aspect of the present invention, there is provided a transdermal spray formulation wherein the transdermal spray formulation comprises a pharmaceutically active agent; VP/VA copolymer and a non-aqueous vehicle.

The non-aqueous vehicle preferably comprises at least about 60% by weight of the formulation.

The transdermal spray formulation may also comprise an anti-nucleating agent.

The transdermal spray formulation may also comprise a penetration enhancer.

In another aspect of the invention there is provided a method of administering a pharmaceutically active agent comprising spraying the transdermal formulation of the invention onto the skin of a subject in need thereof.

In another aspect of the present invention, there is provided a method of forming a pharmaceutically active film comprising spraying a transdermal formulation comprising an effective amount of a pharmaceutically active agent, VP/VA copolymer and a non-aqueous vehicle on the skin of a subject in need thereof.

The present invention provides transdermal drug delivery formulations. Specifically, the present invention provides non-aqueous spray formulations for transdermal drug delivery. More specifically, the invention relates to spray formulations for delivering a pharmaceutically active agent to the skin. In addition to the pharmaceutically active agent, formulations of the invention comprise a VP/VA copolymer and a non-aqueous vehicle that preferably volatilizes at mammalian body temperature. Upon application, the present formulations quickly dry to produce a film patch containing the active agent in finely dispersed particles. The film patch is easily washable in water. In some embodiments, patches produced according to the invention provide improved bioavailability of the active agent compared to conventionally utilized methods of topical administration.

### DETAILED DESCRIPTION OF THE INVENTION

As used herein, a “pharmaceutically active agent” refers to an agent that produces a biological effect in in vitro or in vivo systems. The term is intended to include compounds affecting at least one of any therapeutic, prophylactic, pharmacological or physiological response in a subject. More specifically, any active agent that is capable of producing a pharmacological response, either localized or systemic, is within the contemplation of the invention. It should be noted that the active agents might be used singularly or as a mixture of two or more agents or drugs.

As will be understood by those of skill in the art, suitability for transdermal administration of a particular pharmaceutically active agent requires consideration of several factors. For example, prior to incorporating a pharmaceutically active agent in the present formulations, the agent should be evaluated with respect to its permeability through the skin, potential for skin irritation or allergic reaction, pharmacokinetic properties, pharmacodynamic properties, therapeutic window and whether metabolic responses in vivo are consistent with continuous administration.

Non-limiting examples of suitable pharmaceutically active agents that may be used in the present transdermal spray formulations may include, but are not limited to, anti-inflammatory drugs, analgesics, antiarthritic drugs, antispasmodics, antidepressants, antipsychotics, tranquilizers, antianxiety drugs, narcotic antagonists, antiparkinsonism agents, cholinergic agonists, chemotherapeutic drugs, immunosuppressive agents, antiviral agents, antibiotic agents, appetite suppressants, antiemetics, anticholinergics, antihistaminics, antimigraine agents, coronary, cerebral or peripheral vasodilators, hormonal agents, contraceptives, antithrombotic agents, diuretics, antihypertensive agents, cardiovascular drugs and opioids. Suitable pharmaceutically active agents include both those that are soluble in aqueous media as well as those soluble in non-aqueous media. In accordance with an embodiment of the present invention, the pharmaceutically active agent is suitably selected from one or more of the group consisting of estradiol, testosterone, oxybutynin, buprenorphine, and fentanyl. Particularly preferred among these suitable compounds is estradiol.

The pharmaceutically active agents of the present invention may be present in an amount up to about 40% by weight of the formulation. Estradiol formulations suitably comprise about 1% to about 5% of estradiol by weight of the formulation.

The pharmaceutically active agents contained in the present formulation may suitably be included in a variety of forms, depending on the solubility and release characteristics desired. Non-limiting examples of suitable forms include neutral molecules, components of molecular complexes, and pharmaceutically acceptable salts, free acids or bases, or quaternary salts of the same, or as combinations of these. Simple derivatives of drugs such as pharmaceutically acceptable ethers, esters, amides which have desirable retention and release characteristics, and which are easily metabolized at body pH and temperature, may be employed. Enzymes, proactive forms or pro-drugs are also suitable for use in the present invention.

The formulations of the present invention comprise VP/VA copolymers. The term “VP/VA” or “vinyl pyrrolidone/vinyl acetate” refers to a copolymer, containing vinylpyrrolidone (also referred to as N-vinylpyrrolidone, N-vinyl-2-pyrrolidone and N-vinyl-2-pyrrolidinone) as a monomeric unit. The copolymer vinylpyrrolidone-vinyl acetate is generally known in the pharmaceutical industry under the designations Copolyvidon(e), Copolyvidonum or VP-VA (or VP/VA as used herein). VP/VA series products play a good role in film-former. Its hygroscopicity decreases with the increase of the proportion of vinylacetate in the molecule. This property of VP/VA is extremely useful as it works in sprays and lotions. Also, VP/VA copolymers are primary film formers for a variety of products which demand different degrees of water resistance including aerosol, aqueous, and organic solvent systems. These polymers exhibit film flexibility, good adhesion, luster, water remoistenability, and hardness.

The VP/VA copolymer may be present in an amount between about 0.1% to about 20% by weight of the formulation. In another embodiment, the VP/VA copolymer may be present in an amount between about 0.1% by weight to about 5% by weight of the formulation. In another embodiment, the VP/VA copolymer may be present in an amount between about 0.1% by weight to about 2% by weight of the formulation.

The VP/VA copolymer may comprise any proportion of vinylpyrrolidone to vinyl acetate. Preferably the VP/VA copolymer may comprise from 50 to 70 weight % vinylpyrrolidone. In one embodiment, the VP/VA copolymer comprises 60 weight % vinylpyrrolidone.

Preferred VP/VA copolymers may have a K value of between 26 and 38. The preferred VP/VA copolymers have a K value of between 26 and 34.

One suitable VP/VA copolymer is VA64 (powder), comprising 60% vinylpyrrolidone and 40% vinyl acetate, and having a K value of between 26 and 34.

The formulations of the present invention also comprise a non-aqueous vehicle. As used herein, “non-aqueous vehicle” is intended to refer to a vehicle that is substantially water-free. “Substantially water-free,” as used herein, means that water comprises less than about 10% by weight of the total vehicle. Suitably, water comprises less than about 5% of the total vehicle by weight. Most suitably, water comprises less than about 1% of the total vehicle by weight. Vehicles suitably used in accordance with the present invention are non-aqueous solvents that are volatile at mammalian skin temperature, i.e, about 33° C. to about 35° C. Upon application to the skin, the non-aqueous vehicle evaporates, leaving a film of polymer in which the active agent is dispersed as fine particles available for transdermal absorption. Non-limiting examples of suitable non-aqueous vehicles include the solvents ethanol, acetone and methylal, and mixtures thereof.

In accordance with the invention, the type and amount of non-aqueous vehicle used for a given formulation will depend upon several factors, including the solubility of the pharmaceutically active agent. Particularly suitable non-aqueous vehicles solublilize both the pharmaceutically active agent and the VP/VA copolymer.

The non-aqueous vehicle used in the present formulations should be present in an amount from at least about 60% by weight of the formulation. In some embodiments, the non-aqueous vehicle comprises at least about 70%, at least about 80% or at least about 90% by weight of the formulation.

The formulations of the present invention may also comprise additional components, such as anti-nucleating agents and/or penetration enhancers. As used herein, the term “anti-nucleating agent” refers to any material included in the formulation to prevent crystallization of the pharmaceutically active agent from the non-aqueous vehicle. Suitably, the anti-nucleating agent should be present in an amount from about 1% to about 10% of the formulation by weight. In a preferred embodiment, the anti-nucleating agent comprises about 5% of the formulation by weight. A suitable anti-nucleating agent useful in the present invention is a polyvinylpyrrolidone (PVP). The term “polyvinylpyrrolidone” or “PVP” refers to a polymer, either a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. The cross-linked homopolymer is insoluble and is generally known in the pharmaceutical industry under the designations polyvinylpolypyrrolidone, crospovidone and PVP.

A suitable PVP for use in the present invention is known in the art as PVP K-30. Suitably, PVP K-30 is included in an amount from about 1% to 10% of the formulation by weight.

In an embodiment, the VP/VA copolymer may act as an anti-nucleating agent, in which case an additional anti-nucleating agent may be unnecessary.

The present formulations may also comprise agents known to accelerate the delivery of the pharmaceutically active agents through the skin. These agents have been referred to as penetration or permeation enhancers, accelerants, adjuvants and absorption promoters, and are collectively referred to herein as “penetration enhancers.” Penetration enhancers are suitably provided in an amount from about 0.01% to about 5.0% of the formulation.

Examples of penetration enhancers suitable for use in the present invention are monohydric alcohols such as ethanol and isopropyl, butyl and benzyl alcohols, or dihydric alcohols such as ethylene glycol, diethylene glycol, or propylene glycol, dipropylene glycol and trimethylene glycol, or polyhydric alcohols such as glycerin, sorbitol and polyethylene glycol, polyethylene glycol ethers of aliphatic alcohols (such as cetyl, lauryl, oleyl and stearyl) including polyoxyethylene (4) lauryl ether, polyoxyethylene (2) oleyl ether and polyoxyethylene (10) oleyl ether and polyoxyethylene alkyl ethers; vegetable, animal and fish fats and oils such as olive and castor oils, squalene, and lanolin; fatty acid esters such as propyl oleate, decyl oleate, isopropyl palmitate, glycol palmitate, glycol laurate, dodecyl myristate, isopropyl myristate and glycol stearate; fatty acid alcohols such as oleyl alcohol and its derivatives; fatty acid amides such as oleamide and its derivatives; urea and urea derivatives such as allantoin; polar solvents such as dimethyllaurylamide, dodecylpyrrolidone, isosorbitol, salicylic acid; amino acids and higher molecular weight aliphatic surfactants such as lauryl sulfate salts and esters of sorbitol and sorbitol anhydride such as polysorbate 20, which is commercially available under the trademark TWEEN 20, as well as other polysorbates such as 21, 40, 60, 61, 65, 80, 81, and 85. Other enhancers include oleic and linoleic acids, ascorbic acid, panthenol, butylated hydroxytoluene, tocopherol, tocopherol acetate, tocopheryl linoleate. Particularly suitable penetration enhancers useful in the present invention include menthol, dimethylisosorbide, glycerylmono-oleate and myristyl lactate.

In an embodiment, the non-aqueous vehicle may act a penetration enhancer, in which case an additional penetration enhancer may be unnecessary.

The formulations of the present invention are generally prepared as follows. The VP/VA copolymer is initially dissolved in the non-aqueous vehicle, followed by addition of the pharmaceutically active agent. If necessary, the solution may be sonicated until the pharmaceutically active agent has dissolved. As will be understood by those of skill in the art, additional or alternative means of dissolving the active agent may be used.

The present invention further encompasses a method of administering transdermal spray formulations. The term “administering”, as used herein, is intended to mean any mode of application to a tissue of a subject which results in the physical contact of the formulation with an anatomical site or surface area. The term “subject” is intended to include all warm-blooded mammals, preferably humans.

The term “therapeutically effective amount”, as used herein with reference to the pharmaceutically active agent, is intended to mean the amount of active agent sufficient to produce the desired effect, local or systemic, when applied topically over the duration of intended use. In some embodiments, the film is allowed to remain on the skin for about 24 hours. Typically, the pharmaceutically active agent is delivered in a controlled release manner.

With respect to particular active agents, therapeutically effective amounts are known in the literature or may be determined by methods known in the art. Typically, effective amounts range from about 0.1 mg to about 2,100 mg, depending on the active agents chosen and the site of application. The only upper limit on the amount of the active agent is that the composition should remain substantially free of crystals and that the amount of solvent required for dissolving the active agent should not inhibit the patch-forming properties of the formulation.

As will be understood by those of skill in the art, therapeutic dosage and dosage unit amounts can be estimated by in vitro flux data. The concentration as well as the quantity of the active agent per unit area, namely per square or cubic centimeter, can be varied independently in order to achieve the desired therapeutic effect. The thickness of the film patch left on the skin can also be varied. In some embodiments, a metered dose spray apparatus may be used to apply the formulation. A metered dose spray apparatus, when used at a fixed distance, allows for the formation of a uniform thin film on the skin. In certain embodiments, the metered dose spray apparatus can be a non-aerosol spray apparatus.

The invention further provides a method of forming a pharmaceutically active film comprising spraying a transdermal formulation in accordance with the invention on the skin of a subject in need thereof. As used herein, the term “film” refers to a polymer film containing a pharmaceutically active agent that forms on the skin after application and subsequent drying. As described herein above, a film is formed upon volatilization of the non-aqueous vehicle shortly after contacting the skin. Preferably, the film coating is formed in about 60 seconds or less.

The following example is provided to assist in a further understanding of the invention. The particular materials and conditions employed are intended to be further illustrative of the invention and are not limiting upon the reasonable scope thereof.

### EXAMPLE

Formulation for Transdermal Spray for Testosterone

A transdermal spray formulation comprising testosterone as the active agent was prepared by first dissolving the VP/VA in ethanol/acetone and subsequently adding and dissolving the active agent, followed by the addition of the remaining ingredients. The resulting formulation contained the following components in the following amounts:

| Ingredient | Quantity/batch (% w/w) |
|---|---|
| Testosterone | 16.66% |
| VP/VA copolymer | 0.42% |
| Ethanol | 70.48% |
| Acetone | 12.44% |

While the present invention has now been described and exemplified with some specificity, those skilled in the art will appreciate the various modifications, including variations, additions and omissions that may be made in what has been described. Accordingly, it is intended that these modifications also be encompassed by the present invention.

All patents, publications and references cited herein are hereby fully incorporated by reference. In case of conflict between the present disclosure and incorporated patents, publications and references, the present disclosure should control and that the scope of the present invention be limited solely be the broadest interpretation that lawfully can be accorded the appended claims.

1. A transdermal spray formulation comprising:
a) a pharmaceutically active agent;
b) 0.1% to 2.0% by weight VP/VA copolymer;
c) at least 60% by weight of a non-aqueous solvent; and
d) optionally a penetration enhancer, which, if present, is present in an amount of 0.01% to 5.0% by weight of the composition.

2. A transdermal spray formulation according to claim 1, wherein the pharmaceutically active agent is provided in a therapeutically effective amount.

3. A transdermal spray formulation according to claim 1, further comprising an anti-nucleating agent.

4. A transdermal spray formulation according to claim 3, wherein the anti-nucleating agent is a polyvinylpyrrolidone polymer or copolymer.

5. A transdermal spray formulation according to claim 3, wherein the anti-nucleating agent comprises from about 1% to about 10% by weight of the formulation.

6. A transdermal spray formulation according to claim 1, wherein the penetration enhancer is a monohydric alcohol such as ethanol, isopropyl, butyl or benzyl alcohol; a dihydric alcohol such as ethylene glycol, diethylene glycol propylene glycol, dipropylene glycol or trimethylene glycol; a polyhydric alcohol such as glycerin, sorbitol or polyethylene glycol; a polyethylene glycol ether of an aliphatic alcohol, such as cetyl, lauryl, oleyl or stearyl, including polyoxyethylene (4) lauryl ether, polyoxyethylene (2) oleyl ether, plyoxyethylene (10) oleyl ether or polyoxyethylene alkyl ether; vegetable, animal or fish fats or oil such as olive and castor oils, squalene or lanolin; a fatty acid ester such as propyl oleate, decyl oleate, isopropyl palmitate, glycol palmitate, glycol laurate, dodecyl myristate, isopropyl myristate or glycol stearate; a fatty acid alcohol such as oleyl alcohol and derivatives thereof, a fatty acid amide such as oleamide and derivatives thereof; urea and urea derivatives such as allantoin; a polar solvent such as dimethyllaurylamide, dodecylpyrrolidone, isosorbitol, or salicylic acid; an amino acid; a higher molecular weight aliphatic surfactant such as lauryl sulfate salts or esters of sorbitol and sorbitol anhydride; polysorbates 20. 21. 40, 60, 61. 65, 80, 81, or 85; oleic and linoleic acids, ascorbic acid, panthenol, butylated hydroxytoluene, tocopherol, tocopherol acetate, tocopheryl linoleate, menthol, dimethylisosorbide, glycerylmono-oleate or myristyl lactate.

7. A transdermal spray formulation according to claim 1, wherein the penetration enhancer is selected from the group consisting of menthol, dimethylisosorbide, glycerylmono-oleate and myristyl lactate.

8. A transdermal spray formulation according to claim 1, wherein the non-aqueous solvent is volatile and evaporates at mammalian skin temperature.

9. A transdermal spray formulation according to claim 1, wherein the non-aqueous vehicle is one or more of ethanol, acetone and methylal.

10. A transdermal spray formulation according to claim 1, wherein the pharmaceutically active agent is one or more of the following classes; anti-inflammatory drugs, analgesics, anti-arthritic drugs, antispasmodics, antidepressants, anti-psychotics, tranquillisers, anti-anxiety drugs, narcotic antagonists, antiparkinsonian agents, cholinergic agonists, chemotherapeutic drugs, immunosuppressive agents, antiviral agents, antibiotic agents, appetite suppressants, anti-emetics, anti-cholinergics, antihistaminics, anti-migraine agents, coronary, cerebral or peripheral vasodilators, hormonal agents, contraceptives, anti-thrombotic agents, diuretics, antihypertensive agents, cardiovascular drugs and opioids.

11. A transdermal spray formulation according to claim 1, wherein the pharmaceutically active agent is one or more of estradiol, testosterone, oxybutynin, buprenorphine and fentanyl.

12. A transdermal spray formulation according to claim 1, wherein the pharmaceutically active agent is estradiol.

13. A transdermal spray formulation according to claim 12, wherein the estradiol is present in an amount from about 1% to about 5% by weight of the formulation.

14. A transdermal spray formulation according to claim 1, wherein the pharmaceutically active agent is testosterone.

15. A transdermal spray formulation according to claim 14, wherein the testosterone is present in an amount up to about 16.66% by weight of the formulation.

16. A transdermal spray formulation according to claim 1 for forming a patch on the skin of a subject, wherein the non-aqueous solvent comprises ethanol, methylal or acetone or mixtures thereof; and wherein the optional penetration enhancer, when present, is different to the non-aqueous solvent.

17. A transdermal spray formulation according to claim 16, wherein the non-aqueous solvent comprises ethanol.

18. A method of administering a pharmaceutically active agent, comprising spraying a transdermal formulation according to claim 1 onto the skin of a subject in need thereof.

19. A method according to claim 18, wherein the non-aqueous solvent volatizes upon contact with the skin, forming a film comprising the VP/VA copolymer and the pharmaceutically active agent.

20. A method of forming a pharmaceutically active film comprising spraying a transdermal formulation according to claim 1 on the skin of a subject in need thereof.
